(19) United States
(12) Patent Application Publication
(10) Pub. No.: US 2007/0292397 A1
(43) Pub. Date: Dec. 20, 2007

(54) METHOD FOR THE DETECTION AND NEUTRALIZATION OF BACTERIA

(76) Inventors: Amy K. McNulty, San Antonio, TX (US); Kristine Kieswetter, San Antonio, TX (US); Daniel C. Wadsworth JR., San Antonio, TX (US)

Correspondence Address: KINETIC CONCEPTS, INC.;LEGAL DEPARTMENT INTELLECTUAL PROPERTY, P.O. BOX 659508, SAN ANTONIO, TX, 78265, US

(21) Appl. No.: 11/764,604

(22) Filed: Jun. 18, 2007

Related U.S. Application Data
(60) Provisional application No. 60/814,725, filed on Jun. 19, 2006.

(51) Int. Cl. A61K 35/76; C12Q 1/70; G01N 33/554

(52) U.S. Cl. 424/93.6; 435/7.32; 435/5; 424/445

(57) ABSTRACT

The present invention relates to the identification of bacteria present at the site of infection and the treatment of the infection using bacteriophage. In certain embodiments, the present invention provides methods and compositions for treating bacterial infections by identifying at least one bacteria species in the infection based on its interaction with bacteria-specific aptamers, selecting one or more bacteriophage that infect the identified bacteria species, and administering an effective amount of the bacteriophage to the subject to treat the infection.

This application claims priority to U.S. Provisional Patent Application No. 60/814,725, filed Jun. 19, 2006, the entire content of which is incorporated herein by reference.

### BACKGROUND OF THE INVENTION

1. Field of the Invention

The present invention relates generally to treating bacterial infections. More particularly, it concerns the identification of bacteria present at the site of infection, and the treatment of the infection using bacteriophage.

2. Description of Related Art

Bacterial infection is the detrimental colonization of a host organism by one or more bacterial species. Such infections are commonly treated with antibiotics and/or antiseptics, often without identifying which bacteria are present and, in some cases, without even confirming that bacteria are present at all. When an identification of the bacteria is attempted, microbial culture is a commonly used approach. With this approach, a sample is taken from the potentially diseased tissue or fluid and is contacted with a growth medium or panel of growth mediums. The size, color, shape, and form of the bacterial colonies that form on the growth medium can be characteristic of particular bacterial species. In addition, the ability of bacteria to either grow, not grow, or produce a characteristic color on certain types of growth medium is also used to identify the bacteria present in the sample. Drawbacks to diagnostic techniques that require microbial culture include the time required to grow the bacteria and the fact that certain microbes, such as Mycobacterium, are difficult to culture.

Another tool that may be used independently, or in combination with microbial culture, is microscopy. Microscopy may be used to identify bacteria species based on their morphology. Additionally, microscopy may be used in combination with biochemical staining techniques to identify bacteria. These staining techniques may employ dyes such as in the Giemsa stain, Gram stain, and acid-fast stain techniques. Biochemical staining techniques may also employ antibodies specific to particular bacteria species.

Treatment of bacterial infections typically involves the use of antibiotics. Certain antibiotics are more effective in treating certain bacteria species. In some cases, bacteria cultured from an infection are exposed to a panel of antibiotics to determine the antibiotics to which the bacteria are sensitive or resistant. This, however, can take several days. To avoid the delay that may be associated with the identification of bacteria, antibiotic treatment is often prescribed without a specific identification of the bacteria or even without confirming that the infection is caused by bacteria. This can result in treatments that are unnecessary or not appropriate for the bacteria causing the infection. The unnecessary or inappropriate use of antibiotics also promotes the selection of drug-resistant bacteria strains. Other obstacles associated with antibiotic therapies include adverse reactions that some antibiotics cause in certain patients, and the occurrence of antibiotic resistant bacteria strains.

### SUMMARY OF THE INVENTION

In one embodiment, the present invention provides a method of detecting bacteria in an infection comprising: (a) obtaining a sample from an infection site in a subject; (b) contacting the sample with one or more bacteria-specific aptamers; and (c) detecting an interaction between the bacteria-specific aptamers and bacteria present in the sample, wherein the bacteria in the infection are detected. In certain aspects of the invention, the method further comprises identifying at least one bacteria species in the sample based on its interaction with the bacteria-specific aptamers. In some aspects of the invention, the method further comprises selecting one or more bacteriophage that infect the identified bacteria species, and administering an effective amount of the bacteriophage to the subject.

In another embodiment, the present invention provides a method of treating an infection comprising: (a) obtaining a sample from an infection site in a subject; contacting the sample with one or more bacteria-specific aptamers; (c) detecting an interaction between the bacteria-specific aptamers and bacteria present in the sample; (d) identifying at least one bacteria species in the sample based on its interaction with the bacteria-specific aptamers; and (e) selecting one or more bacteriophage that infect the identified bacteria species; and (f) administering an effective amount of the bacteriophage to the subject, wherein the infection is treated. In certain embodiments, the method further comprises quantifying the amount of bacteria present in the sample.

In another embodiment, the present invention provides a method for treating a wound or promoting healing of a wound comprising: (a) obtaining a tissue or fluid sample from the wound; (b) contacting the sample with one or more bacteria-specific aptamers; (c) detecting an interaction between the bacteria-specific aptamers and bacteria present in the sample; (d) identifying at least one bacteria in the sample based on its interaction with the bacteria-specific aptamers; and (e) selecting one or more bacteriophage that infect the identified bacteria; and (f) topically administering an effective amount of the bacteriophage to the wound, wherein the wound is treated and/or the healing of the wound is promoted. The wound may be, for example, a surgical wound, an acute wound such as a wound caused by an acute injury, a burn, an ulcer such as a diabetic ulcer or a pressure ulcer.

The methods and compositions of the present invention may be used in the identification and treatment of any bacterial infection including, for example, infections of the skin, soft tissue, muscle, bone, upper digestive tract, lower digestive tract, pulmonary system, cardiovascular system, central nervous system, the eyes, urinary tract, reproductive tract, sinuses, or blood (i.e., sepsis). The subject to be treated according to the present invention may be any organism that is susceptible to bacterial infection including, but not limited to, mammals such as humans, livestock (e.g., cattle, horses, sheep, and swine), and domestic pets (e.g., cats and dogs).

The infection may be assayed either in vitro or in vivo for the presence of bacteria. In certain aspects of the invention, a sample is obtained from at or around the site of infection. The method for obtaining the sample may vary depending on the location of the infection and/or the tissues infected. Medical practitioners will be able to determine which approach is suitable for a given subject's condition. For example, the sample may be obtained by aspiration, biopsy, swabbing, venipuncture, spinal tap, or urine sample.

Numerous bacteria species are capable of causing infection in a host organism. Even bacteria that are generally considered to exist in a mutualistic or commensal relationship with their host may cause an infection if, for example, the host's immune system is compromised or the bacteria gains access to a part of the host organism that is normally sterile. Wounds caused by injury, ulcers (e.g., diabetic ulcers, pressure ulcers), or surgery provide an opportunity for bacterial infection because they provide a breach in the skin or mucus membrane through which bacteria can enter the host. Bacteria species that are commonly recovered from wounds and other infections include Escherichia coli, Proteus species, Klebsiella species, Pseudomonas aeruginosa and other Pseudomonas species, Enterobacter species, Streptococcus pyogenes and other Streptococcus species, Bacteroides species, Prevotella species, Clostridium species, Staphylococcus aureus and other Staphylococcus species. Anaerobic bacteria in particular tend to thrive in decaying tissue and deep wounds, especially if the tissue has a poor blood supply. Disease-causing anaerobes include Clostridia, Peptococci, Peptostreptococci, Bacteroides, Actinomyces, Prevotella, and Fusobacterium. Examples of other bacteria that may infect a host organism include Bacillus, Xanthomonas, Vibrio, Salmonella, Shigella, Erwinia, Rickettsia, Chlamydia, Mycoplasma, Actinomyces, Streptomyces, Mycobacterium, Micrococcus, Lactobacillus, Diplococcus, and Borrelia. An infection may contain one or multiple species of bacteria. In certain aspects of the invention, 1, 2, 3, 4, 5, 6, 7, 8, 9, 10, 11, 12, 13, 14, 15, 16, 17, 18, 19, 20, or any range derivable therein of different bacteria species are identified in the infection.

As mentioned above, the bacteria present in a sample may be detected and identified using bacteria-specific aptamers. Bacteria-specific aptamers are aptamers that specifically bind to a marker accessible on the surface of the bacteria and that distinguishes one bacteria strain, bacteria species, or group of bacteria from another bacteria strain, bacteria species, or group of bacteria. The marker that a bacteria-specific aptamer binds may be, for example, a protein or motif that is unique to a particular bacteria strain, bacteria species, or group of bacteria. In some embodiments, the bacteria-specific aptamer binds to a protease, toxin, or drug-resistance protein such as penicillinase. In one embodiment a bacteria-specific aptamer specifically binds to gram-negative bacteria or gram-positive bacteria. For example, techoic acids, which play a role in adherence, are present only in gram-positive bacteria. Accordingly, an aptamer that specifically binds a techoic acid may be used to detect and identify gram-positive bacteria. As a further example, lipoproteins are only present in gram-negative bacteria. Thus, an aptamer that specifically binds a bacterial lipoprotein may be used to detect and identify gram-negative bacteria. In some aspects of the invention, a bacteria-specific aptamer specifically binds to Escherichia coli, Streptococcus pyogenes, Clostridium perfringens, or Staphylococcus aureus. In certain aspects of the invention 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or any range derivable therein, different bacteria-specific aptamers are used to detect and/or identify the bacteria. By using a panel of different bacteria-specific aptamers, multiple different bacteria may be identified contemporaneously.

To facilitate the detection and/or identification of the bacteria, the bacteria-specific aptamers may be labeled. A variety of methods are know for labeling aptamers. For example, aptamers may be labeled with fluorophores, chromophores, radiophores, enzymatic tags, antibodies, chemiluminescence, electroluminescence, affinity labels, biosensors, molecular beacons, quantum dots, or carbon nanotubes. Examples of fluorophores include, but are not limited to the following: all of the Alexa Fluor® dyes, AMCA, BODIPY® 630/650, BODIPY® 650/665, BODIPY®-FL, BODIPY®-R6G, BODIPY®-TMR, BODIPY®-TRX, Cascade Blue®, CyDyes™, including but not limited to Cy2™, Cy3™, and Cy5™, DNA intercalating dyes, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, and Texas Red®.

In certain embodiments of the invention, the bacteria-specific aptamer is immobilized on a solid support such as, for example, a microsphere, a slide, a chip, a column, or nitrocellulose. In certain aspects of the invention, the microsphere is labeled. In certain embodiments, where a first bacteria-specific aptamer is immobilized on a solid, visualization of the interaction between the bacteria and the aptamer may be achieved with the use of a second, unbound bacteria-specific aptamer, which is labeled with a reporter molecule. The first and second bacteria-specific aptamers may or may not have the same binding specificity.

The visualization of the bacteria-specific aptamers bound to the bacteria may be accomplished by a variety of techniques. The particular technique to be employed will depend in part on the label that is used. In certain aspects of the invention, flow cytometry is used to detect the interaction between the bacteria-specific aptamers and bacteria. In other aspects of the invention, fluorescence microscopy is used to detect the interaction between the bacteria-specific aptamers and bacteria. Microfluidic devices may also be used to process and detect the interaction between the bacteria-specific aptamers and bacteria.

Bacteriophage therapy may be specifically tailored to the particular bacteria present in the infection. For example if Streptococcus, Staphylococcus, and E. coli are present, a cocktail of E. coli phage, Streptococcus phage and Staphylococcus phage may be applied to the infection. In certain aspects of the invention, 1, 2, 3, 4, 5, 6, 7, 8, 9, 10, 11, 12, 13, 14, 15, 16, 17, 18, 19, 20, 30, 40, 50, 60, 70, 80, 90, 100, or any range derivable therein, different phage may are administered to the subject. The different phage may be administered simultaneously or serially. The pharmacist or clinician may combine phage isolates on site to allow personalized therapy. The bacteriophage or bacteriophage cocktail may be applied topically by one of several methods. These methods include topical emulsions or dressings, liquid formulations, intrapleural injections, intravenous application, direct injection into the site of infection, tablets, suppositories, lavage, aerosols, and sprays. In certain aspects of the invention, bacteriophages may be infused into an infected area such as a wound via vacuum instillation. In some embodiments, antibiotics and/or antiseptics may be used in combination with the bacteriophage therapy. In such combination treatments, the bacteriophage, antibiotic, and/or antiseptic may be administered together or they may be administered via different routes and/or at different times.

In further embodiments, the present invention concerns kits for use with the disclosed methods regarding the identification and/or treatment of bacterial infection. Compositions comprising one or more bacteria-specific aptamers may be provided in a kit. Such kits may be used to provide one or more such bacteria-specific aptamers in a ready to use and storable container. In certain aspects of the invention 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or any range derivable therein, different bacteria-specific aptamers are provided in a kit. The container of the kits can generally include at least one vial, test tube, flask, bottle, syringe and/or other container, into which at least one bacteria-specific aptamers may be placed, and/or preferably, suitably aliquoted. Compositions comprising one or more bacteriophage also may be provided in a kit. Such kits may be used to provide one or more such bacteriophage in a ready to use and storable container. In certain aspects of the invention, 1, 2, 3, 4, 5, 6, 7, 8, 9, 10, 11, 12, 13, 14, 15, 16, 17, 18, 19, 20, 30, 40, 50, 60, 70, 80, 90, 100, or any range derivable therein, different phage may be provided in a kit. The container of the kits can generally include at least one vial, test tube, flask, bottle, syringe and/or other container, into which at least one bacteriophage may be placed, and/or preferably, suitably aliquoted. The kits of the present invention may include a means for containing bacteria-specific aptamers, bacteriophage, or any other reagent containers in close confinement for commercial sale. Such containers may include injection and/or blow molded plastic containers into which the desired vials are retained. The bacteria-specific aptamers and the bacteriophage may be packaged together in the same kit or they may be provided in separate kits. The kits may also contain additional reagents such as labeling molecules and solid supports.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. FIG. 1 shows bacteria-specific aptamer 11 immobilized on microsphere 10 both prior to (upper left) and after (lower right) binding to bacteria 12 and the labeled bacteria-specific aptamer 13.

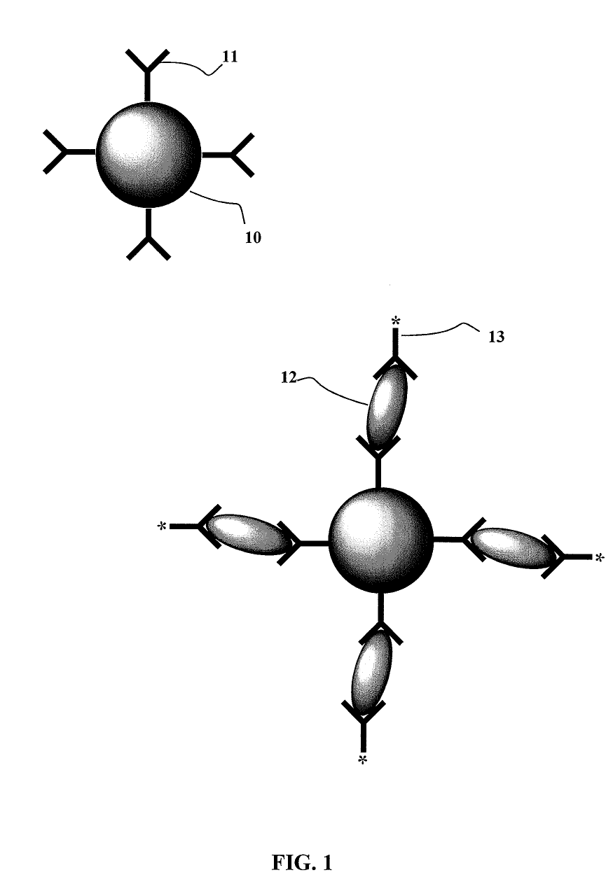

### DESCRIPTION OF ILLUSTRATIVE EMBODIMENTS

A. Detection of Bacteria

The present invention provides methods and compositions for the detection and treatment of bacterial infections. By initial identification of the bacteria associated with an infection, the therapy may be specifically tailored to treat the infection. All bacteria are enclosed by a rigid peptidoglycan cell wall, and the composition of the cell wall varies greatly among different bacteria. This difference provides a basis for the identification of different bacterial species and strains according to the present invention. The peptidoglycan layer is formed from chains of amino sugars, namely N-acetylglucosamine and N-acetylmuramic acid, which are connected by a β-(1,4)-glycosidic bond. Attached to the amino sugars are amino acid chains whose sequence and structure vary among bacterial species. In certain embodiments, the detection involves identification of bacterial species through the use of aptamers that specifically recognize components exposed on the surface of the cell wall.

Examples of markers to which a bacteria-specific aptamer may be targeted include proteases, toxins, drug-resistance proteins, techoic acids, and lipoproteins. Those of ordinary skill in the art would also be able to select a variety of markers suitable for the detection and identification of bacteria using BLAST, which is available on the world wide web at ncbi.nlm.nih.gov/Tools/.

To identify the infective bacteria, one or more proteins or motifs unique to the bacteria are identified. In one embodiment, aptamers specific to the proteins or cell-surface motifs can be selected and used to identify the bacteria. In certain aspects of the invention, detection may also involve the quantification of each species of bacteria present. In addition to the aforementioned embodiments, other methods of visualization for diagnostic purposes may be utilized. For example in vitro analysis may be conducted through the use of quantum dots attached to aptamers. Various sizes of quantum dots could be bound to species specific aptamers. Each size of quantum dot is visible as a slightly different wavelength of light, when excited with light energy. Therefore, use of quantum dots would also allow for the multiplexing diagnostic analyses. Other methods of visualization for diagnostic purposes include, but are not limited to, antibody—fluorescein conjugates, and other antibody—dye or fluorescent components.

B. Aptamers

As mentioned above, aptamers specific to the proteins or cell-surface motifs can be selected and used to identify the bacteria. Aptamers are nucleic acid molecules that may be engineered through repeated rounds of in vitro selection to bind to various targets including, for example, proteins, nucleic acids, cells, tissues, and organisms. Because of their specificity and binding abilities, aptamers have great potential as diagnostic agents. In some cases, aptamers have been shown to be better diagnostic agents than other molecules, such as antibodies. An additional advantage of using aptamers is that mass production does not require either animal or cultured cells. Aptamer synthesis may be conducted through Polymerase Chain Reaction (“PCR”) or oligonucleotide synthesis, and the resulting aptamers are stable at room temperature and have a long shelf life.

Development of aptamers is typically done through SELEX (Systematic Evolution of Ligands by Exponential Enrichment) or variations on the SELEX process. The SELEX process has been described by Turek and Gold, 1990, and in U.S. Pat. Nos. 5,270,163 and 5,475,096, which are incorporated herein by reference. Variations on the SELEX process, such as photo-SELEX, counter-SELEX, chemi-SELEX, chimeric-SELEX, blended-SELEX, and automated-SELEX, have also been reported. Through SELEX, a large population of oligonucleotides is allowed to interact with the target of interest (e.g., a bacteria cell or a protein isolated from the surface of a bacteria cell). Molecules which bind to the target (termed successful) are separated from those that do not bind through one of several techniques. For example, aptamer bound targets may be removed from the population through binding to nitrocellulose, affinity chromatography, etc. The bound aptamers may then be amplified by PCR.

To facilitate the use of the aptamers for diagnostic purposes, the aptamers may be bound to some form of label for visualization. A number of different labels may be used for this purpose such as fluorophores, chromophores, radiophores, enzymatic tags, antibodies, chemiluminescence, electroluminescence, affinity labels, biosensor, or molecular beacons. The method of visualization may differ depending on whether or not the bacterial detection is to be carried out in vivo or in vitro. In one embodiment, aptamers may be bound to carbon nanotubes, which can fluoresce in the near infra red region when excited with red light. The outer surface of single-walled carbon nanotubes may be functionalized, enabling them to modulate their emission when specific biomolecules are adsorbed. In certain embodiments, dyes or fluorophores may be incorporated into the aptamer or encapsulated in lipid bilayers with an aptamer bound to the outside of the bilayer. In some aspects, a quencher molecule may also be incorporated into the aptamer or encapsulated in lipid bilayers with an aptamer bound to the outside of the bilayer. Binding of the labeled aptamer to its specific bacteria will allow for visualization.

An approach involves the multiplexing of microspheres. Microspheres, such as those from Luminex Corporation or Bio-Rad may be coupled to specific aptamers. Each type of bacteria-specific aptamer would be coupled to a bead having slightly different fluorescent properties. Mixtures of bead/aptamers would then be incubated with the suspected infected sample. Bacteria would bind to their specific aptamers. A second incubation with, for example, biotinylated aptamers would allow visualization following streptavidin incubation. The beads may be “read” in a dual laser, flow cytometer (i.e Luminex Technology). A classification laser would allow classification of the bead—aptamer type (e.g. Staphylococcus aptamer). The second, reporter laser would allow quantification of the bacteria present, via reading of the intensity of the streptavidin signal. By this technology up to one hundred or more bacteria may be identified and quantified during a single analysis. The Luminex technology is described, for example, in U.S. Pat. Nos. 5,736,330, 5,981,180, and 6,057,107, all of which are specifically incorporated by reference.

C. Protein Techniques

In some embodiments, the present invention employs methods of isolating proteins from bacteria. Isolated proteins that are unique to a particular bacteria species or strain may then be used in a method, such as SELEX, to engineer aptamers that specifically bind to the protein. Methods of separating proteins are well known to those of skill in the art and include, but are not limited to, various kinds of chromatography (e.g., anion exchange chromatography, affinity chromatography, sequential extraction, and high performance liquid chromatography).

In one embodiment the present invention employs two-dimensional gel electrophoresis to separate proteins from a biological sample into a two-dimensional array of protein spots. Two-dimensional electrophoresis is a useful technique for separating complex mixtures of molecules, often providing a much higher resolving power than that obtainable in one-dimension separations. Two-dimensional gel electrophoresis can be performed using methods known in the art (See, e.g., U.S. Pat. Nos. 5,534,121 and 6,398,933). Typically, proteins in a sample are separated first by isoelectric focusing, during which proteins in a sample are separated in a pH gradient until they reach a spot where their net charge is zero (i.e., isoelectric point). This first separation step results in a one-dimensional array of proteins. The proteins in the one-dimensional array are further separated using a technique generally distinct from that used in the first separation step. For example, in the second dimension proteins may be further separated by polyacrylamide gel electrophoresis in the presence of sodium dodecyl sulfate (SDS-PAGE). SDS-PAGE allows further separation based on the molecular mass of the protein.

Proteins in the two-dimensional array can be detected using any suitable methods known in the art. Staining of proteins can be accomplished with colorimetric dyes (e.g., coomassie), silver staining, or fluorescent staining (Ruby Red; SyproRuby). As is known to one of ordinary skill in the art, spots or protein patterns generated can be further analyzed. For example, proteins can be excised from the gel and analyzed by mass spectrometry. Alternatively, the proteins can be transferred to an inert membrane by applying an electric field and the spot on the membrane that approximately corresponds to the molecular weight of a marker can be analyzed by mass spectrometry.

In certain embodiments the present invention employs mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). The “time of flight” of the ion before detection by an electrode is a measure of the mass-to-charge ratio (m/z) of the ion. Mass spectrometry (MS), because of its extreme selectivity and sensitivity, has become a powerful tool for the quantification of a broad range of bioanalytes including pharmaceuticals, metabolites, peptides and proteins.

Matrix-assisted laser desorption ionization-time of flight mass spectrometry (MALDI-TOF MS) is a type of mass spectrometry in which the analyte substance is distributed in a matrix before laser desorption. MALDI-TOF MS has become a widespread analytical tool for peptides, proteins and most other biomolecules (oligonucleotides, carbohydrates, natural products, and lipids). In combination with 2D-gel electrophoresis, MALDI-TOF MS is particularly suitable for the identification of protein spots by peptide mass fingerprinting or microsequencing.

In MALDI-TOF analysis, the analyte is first co-crystallized with a matrix compound, after which pulse UV laser radiation of this analyte-matrix mixture results in the vaporization of the matrix which carries the analyte with it. The matrix therefore plays a key role by strongly absorbing the laser light energy and causing, indirectly, the analyte to vaporize. The matrix also serves as a proton donor and receptor, acting to ionize the analyte in both positive and negative ionization modes. A protein can often be unambiguously identified by MALDI-TOF analysis of its constituent peptides (produced by either chemical or enzymatic treatment of the sample).

Another type of mass spectrometry is surface-enhanced laser desorption ionization-time of flight mass spectrometry (SELDI-TOF MS). Whole proteins can be analyzed by SELDI-TOF MS, which is a variant of MALDI-TOF MS. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The SELDI-TOF MS approach to protein analysis has been implemented commercially (e.g., Ciphergen).

Tandem mass spectrometry (MS/MS) is another type of mass spectrometry known in the art. With MS/MS analysis ions separated according to their m/z value in the first stage analyzer are selected for fragmentation and the fragments are then analyzed in a second analyzer. Those of skill in the art will be familiar with protein analysis using MS/MS, including QTOF, Ion Trap, and FTMS/MS. MS/MS can also be used in conjunction with liquid chromatography via electrospray or nanospray interface or a MALDI interface, such as LCMS/MS, LCLCMS/MS, or CEMS/MS.

In addition to the methods described above, other methods of protein separation and analysis known in the art may be used in the practice of the present invention. The methods of protein of protein separation and analysis may be used alone or in combination.

In one embodiment the present invention employs two-dimensional gel electrophoresis to separate proteins into a two-dimensional array of protein spots. Two-dimensional electrophoresis is a useful technique for separating complex mixtures of molecules, often providing a much higher resolving power than that obtainable in one-dimension separations. Two-dimensional gel electrophoresis can be performed using methods known in the art (See, e.g., U.S. Pat. Nos. 5,534,121 and 6,398,933). Typically, proteins in a sample are separated first by isoelectric focusing, during which proteins in a sample are separated in a pH gradient until they reach a spot where their net charge is zero (i.e., isoelectric point). This first separation step results in a one-dimensional array of proteins. The proteins in the one-dimensional array are further separated using a technique generally distinct from that used in the first separation step. For example, in the second dimension proteins may be further separated by polyacrylamide gel electrophoresis in the presence of sodium dodecyl sulfate (SDS-PAGE). SDS-PAGE allows further separation based on the molecular mass of the protein.

Proteins in the two-dimensional array can be detected using any suitable methods known in the art. Staining of proteins can be accomplished with calorimetric dyes (e.g., coomassie), silver staining, or fluorescent staining (Ruby Red; SyproRuby). As is known to one of ordinary skill in the art, spots or protein patterns generated can be further analyzed. For example, proteins can be excised from the gel and analyzed by mass spectrometry. Alternatively, the proteins can be transferred to an inert membrane by applying an electric field and the spot on the membrane that approximately corresponds to the molecular weight of a marker can be analyzed by mass spectrometry.

In certain embodiments the present invention employs mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). The “time of flight” of the ion before detection by an electrode is a measure of the mass-to-charge ratio (m/z) of the ion. Mass spectrometry (MS), because of its extreme selectivity and sensitivity, has become a powerful tool for the quantification of a broad range of bioanalytes including pharmaceuticals, metabolites, peptides, and proteins. Modifications of MS have been developed and may be employed in the isolation and identification of proteins. These include, for example, matrix-assisted laser desorption ionization-time of flight mass spectrometry (MALDI-TOF MS), surface-enhanced laser desorption ionization-time of flight mass spectrometry (SELDI-TOF MS), and tandem mass spectrometry (MS/MS).

In addition to the methods described above, other methods of protein separation and analysis known in the art may be used in the practice of the present invention. The methods of protein of protein separation and analysis may be used alone or in combination. Chromatography is used to separate organic compounds on the basis of their charge, size, shape, and solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography that may be used include: adsorption, partition, affinity, gel filtration, and molecular sieve, and many specialized techniques for using them including column, paper, thin-layer, and gas chromatography (Freifelder, 1982).

D. Bacteriophage Therapy

Once bacteria are identified, bacteriophage therapy may be initiated. By initial identification of the bacteria present, the therapy may be specifically tailored to the infection. For example if Streptococcus, Staphylococcus, and E. coli are present, a cocktail of E. coli phage, Streptococcus phage and Staphylococcus phage may be applied to the infection. The pharmacist or clinician may combine phage isolates on site to allow personalized therapy. The bacteriophage or bacteriophage cocktail may be applied topically by one of several methods. These methods include topical emulsions or dressings, liquid formulations, intrapleural injections, intravenous application, tablets and aerosols. Most of these methods have already been tested. Virtually no report of serious complications has been associated with bacteriophage therapy. In addition to the aforementioned methods of bacteriophage delivery, bacteriophages may be infused into an infected area such as a wound via vacuum instillation. This would entail the use of a device such as the V.A.C.® Instill® System. In some embodiments, antibiotics and/or antiseptics may be used in combination with the bacteriophage therapy. In such combination treatments, the bacteriophage, antibiotic, and/or antiseptic may be administered together or they may be administered via different routes and/or at different times.

Bacteriophages are viruses that are capable of infecting bacteria. Phages generally bind to specific molecules on the surface of their target bacteria. Viral DNA is injected into the host bacterium where phage reproduction occurs. Bacteriophages are commonly classified as lytic or lysogenic. Typically only lytic bacteriophages are useful for therapeutic purposes. When lytic phages are used, the ensuing disruption of bacterial metabolism causes the bacteria to lyse. Animal experiments have shown that phage therapy may be superior to antibiotic therapy in treating bacterial infection. For example, antibiotics often kill both harmful and useful bacteria, whereas phage can be more specific in killing only the infectious bacteria. Bacteriophage are self-replicating in bacteria and can penetrate deep into an infection to destroy the bacteria. In addition, bacteriophages are also self-limiting because they require their specific bacterium in order to exist and, in the absence of that bacterium, they are rapidly eliminated. Bacteriophage preparations are also highly stable and easily dispersed in media. They also have a low cost of production and may be stored for long periods of time.

E. Pharmaceutical Preparations

1. Formulations

Pharmaceutical preparations of bacteriophage for administration to a subject are contemplated by the present invention. One of ordinary skill in the art would be familiar with techniques for administering bacteriophage to a subject. Furthermore, one of ordinary skill in the art would be familiar with techniques and pharmaceutical reagents necessary for preparation of these bacteriophage prior to administration to a subject.

In certain embodiments of the present invention, the pharmaceutical preparation will be an aqueous composition that includes the bacteriophage. Aqueous compositions of the present invention comprise an effective amount of a solution of the bacteriophage in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the bacteriophage, its use in the therapeutic compositions is contemplated. Supplementary active ingredients can also be incorporated into the compositions. For human administration, preparations should meet sterility, pyrogenicity, general safety and purity standards as required by the FDA Center for Biologics.

Upon formulation, solutions will be administered in a manner compatible with the dosage formulation and in such amount as is therapeutically effective. The bacteriophage may be administered with other agents that are part of the therapeutic regiment of the subject, such as antibiotic therapy.

2. Dosage

The present invention contemplates administration of bacteriophage for the treatment of bacterial infections. One of ordinary skill in the art would be able to determine the number of bacteriophage to be administered and the frequency of administration in view of this disclosure. The quantity to be administered, both according to number of treatments and dose, may also depend on the subject to be treated, the state of the subject, the location of the infection, the quantity of bacteria present in the infection, and/or the quality of the blood supply to the site of infection. Precise amounts of the therapeutic composition also depend on the judgment of the practitioner and are peculiar to each individual. Frequency of administration could range from 2-6 hours, to 6-10 hours, to 1-2 days, to 1-4 weeks or longer depending on the judgment of the practitioner.

In certain embodiments, it may be desirable to provide a continuous supply of the bacteriophage formulation to the patient. Continuous perfusion of the region of interest (such as a wound) may be preferred. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. The dose of the bacteriophage via continuous perfusion may be equivalent to that given by single or multiple doses, adjusted for the period of time over which the doses are administered.

It may be desirable to combine bacteriophage treatment with other anti-bacterial agents or methods used in the treatment of infections. Such antibacterial agents may be antibiotics or antiseptics. Debriding the site of infection may also be done in combination with the bacteriophage therapy. Combination therapy may be achieved by administering to the subject a single composition or pharmacological formulation that includes both bacteriophage and an additional anti-bacterial agent, or by administering to the subject two distinct compositions or formulations, wherein one composition includes the bacteriophage and the other includes the additional anti-bacterial agent(s). Where two or more distinct compositions or formulations are administered to the subject, bacteriophage may precede or follow the other treatment by intervals ranging from minutes to weeks. It is also contemplated that the distinct compositions or formulations, whether being administered contemporaneously or at intervals, may be administered via different routes of administration. For example, the bacteriophage containing composition may be administered topically while an antibiotic containing composition is administered orally.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,270,163
U.S. Pat. No. 5,475,096
U.S. Pat. No. 5,534,121
U.S. Pat. No. 5,736,330
U.S. Pat. No. 5,981,180
U.S. Pat. No. 6,057,107
U.S. Pat. No. 6,398,933
Freifelder, In: Physical Biochemistry Applications to Biochemistry and Molecular Biology, 2nd Ed. Wm. Freeman and Co., NY, 1982.
Turek and Gold, Science, 249:505-510, 1990.

What is claimed is:

1. A method of detecting bacteria in an infection comprising:
(a) obtaining a sample from an infection site in a subject;
(b) contacting the sample with one or more bacteria-specific aptamers; and
(c) detecting an interaction between the bacteria-specific aptamers and bacteria present in the sample, wherein the bacteria in the infection are detected.

2. The method of claim 1 further comprising identifying at least one bacteria species in the sample based on its interaction with the bacteria-specific aptamers.

3. The method of claim 2 further comprising selecting one or more bacteriophage that infect the identified bacteria species, and administering an effective amount of the bacteriophage to the subject.

4. The method of claim 1, wherein the infection is a skin infection, muscle infection, bone infection, upper digestive tract infection, lower digestive tract infection, pulmonary infection, cardiovascular infection, central nervous system infection, ocular infection, urinary tract infection, reproductive tract infection, or blood infection.

5. The method of claim 1, wherein the sample is obtained by aspiration, biopsy, swabbing, or venipuncture.

6. The method of claim 1, wherein the sample is contacted with between 1 and 10 different bacteria-specific aptamers.

7. The method of claim 1, wherein the sample is contacted with between 10 and 100 different bacteria-specific aptamers.

8. The method of claim 1, wherein the bacteria-specific aptamers are labeled.

9. The method of claim 8, wherein the label is a fluorescent dye, a quantum dot, or a carbon nanotube.

10. The method of claim 1, wherein the bacteria-specific aptamer is immobilized on a solid support.

11. The method of 8, wherein the solid support is a microsphere.

12. The method of claim 1, wherein detecting the interaction between the bacteria-specific aptamers and bacteria present in the sample comprises flow cytometry.

13. The method of claim 1, wherein detecting the interaction between the bacteria-specific aptamers and bacteria present in the sample comprises fluorescence microscopy.

14. The method of claim 2, wherein between 1 and 3 different bacteria species are identified in the sample.

15. The method of claim 2, wherein between 3 and 10 different bacteria species are identified in the sample.

16. The method of claim 2, wherein the one or more bacteria species identified in the sample are of a genus selected from the group consisting of Bacillus, Clostridium, Pseudomonas, Xanthomonas, Vibrio, Bacteroides, Escherichia, Klebsiella, Salmonella, Shigella, Erwinia, Rickettsia, Chlamydia, Mycoplasma, Actinomyces, Streptomyces, Mycobacterium, Micrococcus, Staphylococcus, Lactobacillus, Diplococcus, Streptococcus, and Borrelia.

17. The method of claim 3, wherein between 1 and 5 different bacteriophage are selected.

18. The method of claim 3, wherein one or more of the bacteriophage are specific for one or more of Bacillus, Clostridium, Pseudomonas, Xanthomonas, Vibrio, Bacteroides, Escherichia, Klebsiella, Salmonella, Shigella, Erwinia, Rickettsia, Chlamydia, Mycoplasma, Actinomyces, Streptomyces, Mycobacterium, Micrococcus, Staphylococcus, Lactobacillus, Diplococcus, Streptococcus, or Borrelia.

19. The method of claim 3, wherein the bacteriophage are administered topically to the site of infection.

20. The method of claim 3, wherein the bacteriophage are administered by injection.

21. The method of claim 20, wherein the injection is a direct injection into an infection site.

22. The method of claim 20, wherein the injection is an intrapleural injection or intravenous injection.

23. The method of claim 1, further comprising quantifying the amount of bacteria present in the sample.

24. A method for treating a wound comprising:
(a) obtaining a tissue or fluid sample from the wound;
(b) contacting the sample with one or more bacteria-specific aptamers;
(c) detecting an interaction between the bacteria-specific aptamers and bacteria present in the sample;
(d) identifying at least one bacteria species in the sample based on its interaction with the bacteria-specific aptamers; and
(e) selecting one or more bacteriophage that infect the identified bacteria species; and
(f) topically administering an effective amount of the bacteriophage to the wound, wherein the wound is treated.

25. The method of claim 24, wherein the wound is a surgical wound.

26. The method of claim 24, wherein the wound is an acute wound.

27. The method of claim 24, wherein the wound is a burn.

28. The method of claim 24, wherein the wound is a diabetic ulcer or a pressure ulcer.

29. The method of claim 24, wherein the tissue sample is obtained by debriding the wound.

30. The method of claim 24, wherein the fluid sample is obtained by aspirating, irrigating, or swabbing the wound.

31. The method of claim 24, wherein the topical administration of the bacteriophage comprises applying a topical emulsion or dressing to the wound.

32. The method of claim 24, wherein the topical administration of the bacteriophage comprises applying an aerosol or a spray to the wound.

33. The method of claim 24, wherein the topical administration of the bacteriophage comprises infusion of the wound using vacuum instillation.
